(19) United States
(12) Patent Application Publication
(10) Pub. No.: US 2025/0361324 A1
(43) Pub. Date: Nov. 27, 2025

(54) IGM ANTIBODIES DEGRADING IGG

(71) Applicants: VACCINVENT GMBH, Freiburg im Breisgau (DE); UNIVERSITÄT ULM, Ulm (DE)

(72) Inventor: Hassan JUMAA-WEINACHT, Blaubeuren (DE)

(21) Appl. No.: 18/993,299

(22) PCT Filed: Jul. 28, 2023
(86) PCT No.: PCT/EP2023/071088

§ 371 Date: Jan. 10, 2025

(30) Foreign Application Priority Data
Jul. 28, 2022 (EP) 22187672.5

(51) Int. Cl. C07K 16/42; A61K 39/00; A61P 19/02; A61P 25/00; G01N 33/564

(52) U.S. Cl. CPC C07K 16/42; A61P 19/02; A61P 25/00; G01N 33/564; A61K 2039/505; C07K 2317/92; G01N 2800/102; G01N 2800/285

(57) ABSTRACT

The invention relates to a glycosylated IgM antibody cross-specifically binding to an IgG antibody and a complexing molecule such as DNA, wherein the binding to the IgG antibody and the complexing molecule induces degradation of the IgG antibody. The Kd for the binding affinity of the IgM antibody to the IgG antibody is preferably in the range of 10⁻⁵ to 10⁻⁸. The invention further relates to medical uses of the glycosylated IgM antibody such as the use in the treatment of autoimmune diseases, e.g., systemic lupus erythematosus, rheumatoid arthritis and multiple sclerosis.

### IgM Antibodies Degrading IgG

The invention relates to a glycosylated IgM antibody cross-specifically binding to an IgG antibody and a complexing molecule such as DNA, wherein the binding to the IgG antibody and the complexing molecule induces degradation of the IgG antibody. The K_{d} for the binding affinity of the IgM antibody to the IgG antibody is preferably in the range of 10⁻⁵ to 10⁻⁸. The invention further relates to medical uses of the glycosylated IgM antibody such as the use in the treatment of autoimmune diseases, e.g., systemic lupus erythematosus, rheumatoid arthritis and multiple sclerosis.

The process of antibody generation leads to the formation of infinite antigen binding sites by random rearrangement of gene segments, namely Variable (V), Diversity (D) and Joining (J) segments. The random nature of antibody specificity generation ensures the recognition of a nearly unlimited variety of antigens but inevitably leads to the generation of self-reactive specificities. The majority of early B cells possess autoreactive BCRs and it is believed that the highly autoreactive cells are eliminated from the repertoire by central tolerance, which induces receptor editing by secondary Immunoglobulin (Ig) gene recombination thereby altering the specificity of the autoreactive B cells. If receptor editing fails to replace the autoreactive specificity, the respective autoreactive B cells are eliminated by clonal deletion. If autoreactive B cells escape from central tolerance, they are thought to be functionally silenced as mature B cells by anergy in the periphery. Defects in the elimination of autoreactive B cells are thought to lead to the occurrence of autoimmune diseases such as rheumatoid arthritis (RA) or systemic lupus erythematosus (SLE), which are characterized and diagnosed by the presence of autoantibodies.

Rheumatoid Factor (RF) is one of the first discovered and most studied autoantibodies, already described in the late 1940s as a class of Ig that can bind the Fc portion of IgG (Volkov, Mikhail, Karin Anna Schie, and Diane Woude. 2020, Immunological Reviews 294(1):148-63). Although being one of the best characterized autoantibodies, the role of RF-IgM in immune disease pathogenesis remains elusive. Among the different RF isotypes, IgM-RF is the most clinically used to estimate disease prognosis in Rheumatoid Arthritis (RA), a chronic autoimmune disease marked by infiltration of B and T cells in the synovial membrane of the joints. However, the biological function of RF in disease pathogenesis remains unknown (Volkov, Mikhail, Karin Anna Schie, and Diane Woude. 2020, Immunological Reviews 294(1):148-63).

An important characteristic of RA is the presence of anti-citrullinated protein-IgG (ACPA-IgG) causing inflammation in the synovia. Here, removal of the amino group (NH₃₊) of arginine residues by protein arginine deaminases (PAD4) generates citrullinated proteins mainly localized in joints (Darrah, Erika, and Felipe Andrade. 2018, Current Opinion in Rheumatology 30(1):72-78). Binding of ACPA-IgG to citrullinated proteins seems to lead to the deposition of immune complexes in the joints thereby activating innate immune cells and initiating inflammation.

In this scenario, it is conceivable that RFs acquire pathogenic properties through formation of immune complexes with autoreactive ACPA-IgG antibodies, thereby causing inflammation by stimulating the secretion of proinflammatory cytokines. Interestingly, RA patients are classified into RF positive (RF+) and RF negative (RF−), where the presence of RF indicates a poor prognosis (Smolen, Josef S., Daniel Aletaha, Anne Barton, Gerd R. Burmester, Paul Emery, Gary S. Firestein, Arthur Kavanaugh, Iain B. McInnes, Daniel H. Solomon, Vibeke Strand, and Kazuhiko Yamamoto. 2018. Nature Reviews Disease Primers 4(1):18001).

Thus, there is a need for improved means and methods to control IgG antibodies, in particular to control autoreactive IgG antibodies in immune diseases.

The above technical problem is solved by the embodiments disclosed herein and as defined in the claims.

Accordingly, the invention relates to, inter alia, the following embodiments:
1. A glycosylated IgM antibody cross-specifically binding to an IgG antibody and a complexing molecule, wherein the binding to the IgG antibody and the complexing molecule induces degradation of the IgG antibody.
2. The antibody according to embodiment 1, wherein the K_{d} for the binding affinity of the IgM antibody to the IgG antibody is in the range of 10⁻⁵ to 10⁻⁸, preferably 10⁻⁷.
3. The antibody according to embodiment 1 or 2, wherein at least one complementarity-determining region (CDR) of the IgM antibody binds to the IgG antibody and wherein the glycosylated part of the IgM antibody binds to the complexing molecule.
4. The antibody according to any one of embodiments 1 to 3, wherein the IgG antibody is an autoreactive IgG antibody.
5. The antibody according to any one of embodiments 1 to 4, wherein the complexing molecule is DNA.
6. The antibody according to any one of embodiments 1 to 5, wherein the autoreactive IgG antibody is an anti-citrullinated protein-IgG antibody.
7. The antibody according to any one of embodiments 1 to 6, wherein a first chain comprises CDRs specifically binding to IgG and a second chain comprises CDRs polyreactive binding to IgG.
8. The antibody according to any one of embodiments 1 to 7, for use in medicine.
9. The antibody according to any one of embodiments 1 to 7, for use in the treatment of a subject with increased IgM level, preferably a serum IgM level above 1500 hIgM μm/ml.
10. The antibody according to any one of embodiments 1 to 7, for use in the treatment of a subject with an increased high affinity rheumatoid factor: low affinity rheumatoid factor ratio.
11. The antibody according to any one of embodiments 1 to 7, for use in the treatment of an autoimmune disease or disorder.
12. The antibody for use of embodiment 11, wherein the autoimmune disease or disorder is at least one selected from the group consisting of: Systemic lupus erythematosus, rheumatoid arthritis and multiple sclerosis.
13. The antibody for use of embodiment 12, wherein the autoimmune disease or disorder is rheumatoid arthritis.
14. A method for diagnosing an autoimmune disease or disorder, the method comprising the steps of:
   a) determining a high affinity rheumatoid factor portion and a low affinity rheumatoid factor portion based on the affinity of the rheumatoid factors to IgG antibodies in a sample of a subject; and
   b) diagnosing the subject with an autoimmune disorder based on the high affinity rheumatoid factor portion and the low affinity rheumatoid factor portion determined in a) and/or a ratio thereof.
15. The IgM antibody of any one of embodiments 1 to 7 or the IgM antibody use of any of embodiments 8 to 13, wherein the antibody comprises:

a variable heavy (VH) chain comprising CDR1 sequence as encoded by SEQ ID NO: 5, CDR2 sequence as encoded by SEQ ID NO: 6 and CDR3 sequence as encoded by SEQ ID NO: 7 and a variable light (VL) chain comprising CDR1 sequence as encoded by SEQ ID NO: 2, CDR2 sequence as encoded by GATGCATCC and CDR3 sequence as encoded by SEQ ID NO: 3.
16. The IgM antibody of embodiment 15 or the IgM antibody use of embodiment 15, wherein the antibody comprises:

a variable heavy (VH) chain sequence comprising the amino acid sequence encoded by the sequence as defined by SEQ ID NO: 4 or by a sequence having at least 90% sequence identity to SEQ ID NO: 4, preferably at least 95% sequence identity to SEQ ID NO: 4; and

a variable light (VL) chain sequence comprising the amino acid sequence encoded by the sequence as defined by SEQ ID NO: 1 or by a sequence having at least 90% sequence identity to SEQ ID NO: 1, preferably at least 95% sequence identity to SEQ ID NO: 1.
17. A host cell comprising a polynucleotide having
   a) a sequence as defined by SEQ ID NO: 4 or a sequence having at least 90% sequence identity to SEQ ID NO: 4, preferably at least 95% sequence identity to SEQ ID NO: 4; and/or
   b) a sequence as defined by SEQ ID NO: 1 or a sequence having at least 90% sequence identity to SEQ ID NO: 1, preferably at least 95% sequence identity to SEQ ID NO: 1;

wherein the polynucleotide further encodes an IgM constant region and/or wherein the host cell comprises a further polynucleotide encoding an IgM constant region.
18. A method for producing an IgM antibody, the method comprising the steps of:
   a) culturing the host cell according to embodiment 17,
   b) isolating an IgM antibody.

Accordingly, in one embodiment, the invention relates to a glycosylated IgM antibody cross-specifically binding to an IgG antibody and a complexing molecule, wherein the binding to the IgG antibody and the complexing molecule induces degradation of the IgG antibody.

In one embodiment, the invention relates to a glycosylated IgM antibody binding to an IgG antibody and to a complexing molecule, wherein preferably the binding to the IgG antibody and the complexing molecule induces degradation of the IgG antibody.

The term “IgM antibody”, as used herein, refers to its general meaning in the art and refers to an immunoglobulin that possesses heavy m-chains. Serum IgM exists as a pentamer (or hexamer) in mammals and comprises approximately 10% of normal human serum Ig content. It predominates in primary immune responses to most antigens and is the most efficient complement-fixing immunoglobulin. IgM is also expressed on the plasma membrane of B lymphocytes as membrane-associated immunoglobulin (which can be organized as multiprotein cluster in the membrane). In this form, it is a B-cell antigen receptor, with the H chains each containing an additional hydrophobic domain for anchoring in the membrane. Monomers of serum IgM are bound together by disulfide bonds and a joining (J) chain. Each of the five monomers within the pentamer structure is composed of two light chains (either kappa or lambda) and two heavy chains. Unlike in IgG (and the generalized structure shown above), the heavy chain in IgM monomers is composed of one variable and four constant regions, with the additional constant domain replacing the hinge region. IgM can recognize epitopes on invading microorganisms, leading to cell agglutination. This antibody-antigen immune complex is then destroyed by complement fixation or receptor-mediated endocytosis by macrophages. IgM is the first immunoglobulin class to be synthesized by the neonate and plays a role in the pathogenesis of some autoimmune diseases. Immunoglobulin M is the third most common serum Ig and takes one of two forms: a pentamer (or hexamer under some circumstances) where all heavy chains are identical and all light chains are identical. The membrane-associated form is a monomer (e.g., found on B lymphocytes as B cell receptors) that can form multimeric clusters on the membrane. In some embodiments, the IgM antibody is a monomeric IgM or an oligomeric IgM. In some embodiments, the oligomeric IgM antibody described herein is an antibody selected from the group of: monomeric IgM antibody, dimeric IgM antibody, trimeric IgM antibody, quatromeric IgM antibody, pentameric IgM antibody and hexameric IgM antibody.

The term “glycosylated IgM antibody”, as used herein, refers to an IgM antibody having a glycosylation on at least one glycosylation sites such as the J-chain and/or an N-glycosylation site, preferably on an N-linked glycosylation site. In some embodiments, the IgM antibody has a glycosylation on at least one Asn-linked glycosylation site. In some embodiments, the IgM antibody has a glycosylation on at least one glycosylation site selected from the group consisting of: ASN-46, ASN-209, ASN-272, ASN-279, ASN-440. In some embodiments, the glycosylated IgM antibody described herein is a blood derived antibody. In some embodiments, the glycosylated IgM antibody described herein is recombinantly produced.

The term “binding to” as used in the context of the present invention defines a binding (interaction) of at least two “antigen-interaction-sites” with each other.

The term “cross-specifically binding”, as used herein, refers to binding to at least two binding partners, preferably the at least two binding partners are different, such as an IgG antibody and a complexing molecule. The cross-specificity may also extend to a) a plurality of complexing molecules and/or b) a plurality of IgG antibodies or all IgG antibodies. In some embodiments, the glycosylated IgM antibody binds to the constant region of the IgG antibody/antibodies.

The term “complexing molecule”, as used herein, refers to a molecule that enables a immune-degradable complex formation upon binding to the glycosylated IgM antibody described herein, preferably upon binding to the glycosylated IgM antibody, while the glycosylated IgM antibody described herein is binding to the IgG antibody described herein.

The term “degradation”, as used herein, in the context of an IgG antibody, refers to reduction of functionality, preferably neutralization e.g. by immune cells. Preferably, IgG degradation means diminishing or neutralization of IgG, measured in vivo or in vitro, as described herein in the examples.

As used herein, the term “IgG” has its general meaning in the art and refers to an immunoglobulin that possesses heavy g-chains. Produced as part of the secondary immune response to an antigen, this class of immunoglobulin constitutes approximately 75% of total serum Ig. IgG is the only class of Ig that can cross the placenta in humans, and it is largely responsible for protection of the newborn during the first months of life. IgG is the major immunoglobulin in blood, lymph fluid, cerebrospinal fluid and peritoneal fluid and a key player in the humoral immune response. Serum IgG in healthy humans presents approximately 15% of total protein beside albumins, enzymes, other globulins and many more. There are four IgG subclasses described in human, mouse and rat (e.g. IgG1, IgG2, IgG3, and IgG4 in humans). The subclasses differ in the number of disulfide bonds and the length and flexibility of the hinge region. Except for their variable regions, all immunoglobulins within one class share about 90% homology, but only 60% among classes. IgG1 comprises 60 to 65% of the total main subclass IgG, and is predominantly responsible for the thymus-mediated immune response against proteins and polypeptide antigens. IgG1 binds to the Fc-receptor of phagocytic cells and can activate the complement cascade via binding to C1 complex. IgG1 immune response can already be measured in newborns and reaches its typical concentration in infancy. IgG2, the second largest of IgG isotypes, comprises 20 to 25% of the main subclass and is the prevalent immune response against carbohydrate/polysaccharide antigens. “Adult” concentrations are usually reached by 6 or 7 years old. IgG3 comprises around 5 to 10% of total IgG and plays a major role in the immune responses against protein or polypeptide antigens. The affinity of IgG3 can be higher than that of IgG1. Comprising usually less than 4% of total IgG, IgG4 does not bind to polysaccharides. In the past, testing for IgG4 has been associated with food allergies, and recent studies have shown that elevated serum levels of IgG4 are found in patients suffering from sclerosing pancreatitis, cholangitis and interstitial pneumonia caused by infiltrating IgG4 positive plasma cells. In some embodiments, the IgG antibody described herein is an antibody of at least one subclass selected from the group consisting of: IgG1, IgG2, IgG3, and IgG4.

The inventors found that glycosylated IgM antibodies acting as rheumatoid factors (RF) display neutralizing effects on IgG, thereby leading to faster degradation and diminishing of IgG in vivo. These effector functions are typically independent of the pathogenic or beneficial nature of the target IgG. Without being bound by theory, it appears that degrading RFs, which are also found in healthy individuals, regulate half-life of IgG and control IgG homeostasis and that defects in generating degrading RFs, might be an important trigger for the development of autoimmune diseases. In this scenario, it is conceivable that degrading RF neutralize IgG antibodies by forming large immune complexes together with complexing molecules such as nucleic acids thereby facilitating IgG uptake by immune cells such as phagocytes. Degrading RFs might act as general regulators of IgG by recognizing its constant region. Alternatively or additionally, degrading RFs might act in a distinctive manner by regulating specific IgG idiotypes through the recognition of the individual variable region. In the context of IgG-associated autoimmune disease, this suggests that a highly diverse antibody repertoire is important for the regulation of large spectrum of IgG antibodies targeting individual idiotypes.

This includes the existence of polyreactive neutralizing IgM as opposed to the protective regulatory IgM (Amendt, Timm, and Hassan Jumaa. 2021. The EMBO Journal 40(17)). These findings indicate that one way to possibly reduce the levels of harmful IgG antibodies in circulation would be the use of low affinity RF or total IgM antibodies from healthy individuals as therapeutic antibodies. Interestingly, the generation of idiotype-specific anti-IgG IgM would allow the manipulation of individual IgGs in a specific manner without affecting the entire IgG repertoire.

The current view proposing that autoantibodies develop in consequence to defects in central and peripheral tolerance mechanisms which in healthy conditions should prevent the development of autoreactive B cells (see e.g. Zikherman, Julie, Ramya Parameswaran, and Arthur Weiss. 2012. Nature 489(7414):160-64) is teaching away from the invention.

Accordingly, the invention is at least in part based on the finding that glycosylated IgM antibodies can induce degradation of IgG antibodies as described herein.

In some embodiments described herein, the RF or IgM antibody described herein is an autoreactive antibody or autoantibody.

In some embodiments, the RF of the invention is an IgM antibody, preferably a glycosylated IgM antibody.

In some embodiments described herein, the IgM antibody described herein is a monoclonal antibody. In some embodiments, the antibody described herein is a human, humanized, or chimeric antibody. The production of antibodies can be based, for example, on the immunization of animals, like mice. However, also other animals for the production of antibody/antisera are envisaged within the present invention. For example, monoclonal and polyclonal antibodies can be produced by rabbit, mice, goats, donkeys and the like. Methods for producing and/or altering antibodies are known in the art and described inter alia in laboratory manuals (see Sambrook et al., Molecular Cloning: A Laboratory Manual; Cold Spring Harbor Laboratory Press, 2nd edition (1989) and 3rd edition (2001); Gerhardt et al., 1994, Methods for General and Molecular Bacteriology ASM Press; Lefkovits, 1997, Immunology Methods Manual: The Comprehensive Sourcebook of Techniques; Academic Press); Golemis, 2002, Protein-Protein Interactions: A Molecular Cloning Manual Cold Spring Harbor Laboratory Press).

In certain embodiments, the invention relates to the antibody according to the invention, wherein the K_{d} for the binding affinity of the IgM antibody to the IgG antibody is in the range of about 10⁻⁵ to about 10⁻⁸.

In certain embodiments, the invention relates to the antibody according to the invention, wherein the K_{d} for the binding affinity of the IgM antibody to the IgG antibody is about 10⁻⁷.

As used herein, the term “low affinity” or “binding with low affinity” refers to a K_{d} in the range of about 10⁻⁵ to about 10⁻⁸, preferably of 10⁻⁵ to 10⁻⁸, more preferably of 10⁻⁶ to 10⁻⁸, again more preferably or 10⁻⁶ to 10⁻⁷, for binding affinity. In a very preferred embodiment, low affinity refers to a K_{d} of 10⁻⁷ for binding affinity.

As used herein, the term “high affinity” or “binding with high affinity” refers to a K_{d} in the range of about 10⁻⁹ or a lower K_{d} for binding affinity. The term “K_{d}”, as used herein, refers to the equilibrium dissociation constant of a particular antibody-antigen interaction. The skilled person is well-aware of various methods and assays suitable for determining the K_{d} of an antibody or antigen-binding fragment thereof as provided herein and as encompassed by the present invention. In some embodiments, the K_{d} is determined by bio-layer interferometry. Preferably, the K_{d} is determined by bio-layer interferometry as described herein, especially in the examples and figures of the invention.

The inventors found that low affinity rheumatoid factors display opposite neutralizing effects (compared to high affinity rheumatoid factors) on IgG, thereby leading to faster degradation and diminishing of IgG in vivo. These effector functions are dependent on the affinity of RF-IgM. RF-IgM contribute to faster degradation if their affinity to IgG is low and if they are polyreactive.

As used herein, the term “polyreactive” refers to antibodies binding with low affinity to an antigen. Polyreactive antibodies preferably bind to a variety of structurally unrelated antigens such as free double stranded DNA.

Accordingly, the invention is at least in part based on the finding that IgM antibodies contribute to faster degradation of IgG if their affinity to IgG is low.

In certain embodiments, the invention relates to the antibody according to the invention, wherein at least one CDR of the IgM antibody binds to the IgG antibody.

In certain embodiments, the invention relates to the antibody according to the invention, wherein the glycosylated part of the IgM antibody binds to the complexing molecule.

In certain embodiments, the invention relates to the antibody according to the invention, wherein at least one CDR of the IgM antibody binds to the IgG antibody and wherein the glycosylated part of the IgM antibody binds to the complexing molecule.

In certain embodiments, the invention relates to the antibody according to the invention, wherein a first chain of the IgM antibody comprises CDRs binding to IgG and a second chain binding to the complexing molecule, preferably via a glycosylated chain, preferably via glycosylation of the IgM antibody.

The inventors found that the glycosylated part is particular efficient in binding complexing molecules, if the glycosylated part, e.g., the glycosylation itself binds to the complexing molecule.

In certain embodiments, the invention relates to the antibody according to the invention, wherein at least one CDR of the IgM antibody binds to the IgG antibody.

In certain embodiments, the invention relates to the antibody according to the invention, wherein the glycosylated part, preferably the glycosylated amino acid sequence of the IgM antibody binds or participates in binding to the complexing molecule.

In certain embodiments, the invention relates to the antibody according to the invention, wherein at least one CDR of the IgM antibody binds to the IgG antibody and wherein the glycosylated part of the IgM antibody binds to the complexing molecule.

In certain embodiments, the invention relates to the IgM antibody according to the invention, wherein the IgG antibody is an autoreactive IgG antibody.

The term “autoreactive IgG antibody”, as used herein, refers to an antibody produced by the immune system that is directed against one or more of the subject's own proteins or antigens.

The autoreactive IgG antibody described herein can be involved in the regulation of an endogenous protein or can be characteristic for many autoimmune diseases. In some embodiments, the IgM antibody of the invention binds autoreactive IgG antibodies amongst other IgG antibodies. In some embodiments, the IgM antibody of the invention binds primarily autoreactive IgG antibodies.

Autoreactive IgG antibody are retained in circulation, likely because they play a specific role in maintenance of physiological homeostasis. The IgM antibodies described herein can restore this maintenance upon dysregulation.

Accordingly, the invention is at least in part based on the finding that glycosylated IgM antibodies can regulate and induce degradation of autoreactive IgG antibodies as described herein.

In certain embodiments, the invention relates to the antibody according to the invention, wherein the complexing molecule is a nucleic acid, preferably DNA, more preferably double stranded DNA.

The term “DNA”, as used herein, refers to any complexing molecule comprising deoxyribonucleic acid, typically in the form of a polymer e.g. in double stranded form. The DNA as a complexing molecule can be provided for example in the form of extracellular DNA released by immune cells.

Accordingly, the invention is at least in part based on the finding that binding of the IgM antibody of the invention to DNA results in efficiently degradable complex formation as described herein.

In certain embodiments, the invention relates to the antibody according to the invention, wherein the autoreactive IgG antibody is an anti-citrullinated protein-IgG antibody.

The term “anti-citrullinated protein-IgG antibody”, as used herein, refers to autoantibodies that are directed against peptides and proteins that are citrullinated. These antibodies are typically observed in patients with RA and are believed to play a role in the development and pathology of RA.

In certain embodiments, the invention relates to the antibody according to the invention, wherein a first chain comprises CDRs specifically binding to IgG and a second chain comprises CDRs polyreactive binding to IgG.

In certain embodiments, a first chain of the antibody according to the invention comprises CDRs binding with high affinity to IgG and a second chain of the antibody according to the invention comprises CDRs binding with low affinity to IgG. The term “low affinity” refers to a K_{d} in the range of about 10⁻⁵ to about 10⁻⁸, preferably of 10⁻⁵ to 10⁻⁸, more preferably of 10⁻⁶ to 10⁻⁸, again more preferably or 10⁻⁶ to 10⁻⁷, for binding affinity. Most preferably, low affinity refers to a K_{d} of 10⁻⁷. The term “high affinity” refers to a K_{d} in the range of about 10⁻⁹ or a lower K_{d} for binding affinity.

RF antibodies are mainly linked to RA, nonetheless studies of RF production and incidence have shown that circulating RFs can be found in healthy individuals. Interestingly, RF antibodies which have been studied in RA patients are characterized by extensive somatic mutation and possess high antigen-binding affinity and specificity for IgG acquired during the process of affinity maturation. On the contrary, RFs found in healthy individuals closely resemble natural autoantibodies, a class of autoantibodies with restricted epitope specificity, mainly encoded by germline variable gene segments. As such, the majority of natural autoantibodies is polyreactive and binds self-molecules with low antigen-binding affinity. Similarly, RFs in healthy individuals show no evidence of affinity maturation and isotype switching, suggesting low antigen-binding affinity for IgG (Mageed et al. 1997; Volkov et al. 2020).

In certain embodiments, the invention relates to the antibody according to the invention, for use in medicine.

In certain embodiments, the invention relates to the antibody according to the invention, for use in the treatment of a subject with increased IgM level, preferably a serum IgM level above 1500 hIgM μm/ml. Antibody levels are determined as described herein, especially in the examples.

It is conceivable that high titers of high affinity RFs in the synovial membrane of RA patients acquires pathogenic role because they perpetuate the inflammatory state by stabilizing pathogenic IgG such as anti-citrullinated protein-IgG antibody. The exaggerated function of autoreactive IgG in the joints leads to the formation of immune complexes that may continuously trigger macrophages and complement activation via Fc receptors thereby prolonging inflammation at the synovial membrane.

The treatment with the IgM antibody of the invention may control the general IgG homeostasis e.g. when recognizing the constant region of IgG, whereas they may selectively eliminate pathogenic IgGs when acting at the level of individual idiotypes.

The inventors found that the simultaneous presence of high and low affinity RFs, the effect of the destructive low affinity RF prevails over the protective high affinity RF.

Accordingly, the invention is at least in part based on the finding, that the IgM antibody of the invention can be used in treatment in the (increased) presence of IgG-protective IgM antibodies such as high affinity IgM.

In certain embodiments, the invention relates to the antibody according to the invention, for use in the treatment of a subject with an increased high affinity rheumatoid factor: low affinity rheumatoid factor ratio. The term “increased high affinity rheumatoid factor: low affinity rheumatoid factor ratio” refers to a ratio increased when comparing patients suffering from an autoimmune disease or disorder versus healthy subjects, especially not suffering from an autoimmune disease or disorder.

The inventors found that the simultaneous presence of high and low affinity RFs, the effect of the destructive low affinity RF prevails over the protective high affinity RF. Extending this finding to a more general level, it is conceivable that the ratio between the two RF populations is relevant in the context of autoimmunity.

Accordingly, the invention is at least in part based on the finding that the IgM antibody of the invention can be used to restore a healthy high/low affinity RF ratio.

In certain embodiments, the invention relates to the antibody according to the invention, for use in the prevention of an autoimmune disease or disorder, preferably a chronic autoimmune disease or disorder.

In certain embodiments, the invention relates to the antibody according to the invention, for use in the treatment of an autoimmune disease or disorder, preferably a chronic autoimmune disease or disorder.

In certain embodiments, the invention relates to a method for treatment or prevention an autoimmune disease or disorder, preferably a chronic autoimmune disease or disorder, wherein said method comprises administering the antibody according to the invention to a patient.

The term “treatment” (and grammatical variations thereof such as “treat” or “treating”), as used herein, refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis. In some embodiments, antibodies of the invention are used to delay development of a disease or to slow the progression of a disease.

The term “prevention”, as used herein, relates to the capacity to prevent, minimize or hinder the onset or development of a disorder, disease or condition before its onset.

The fact that low affinity RFs are found in healthy individuals and regulate half-life of IgG suggests that IgG homeostasis is controlled by such RFs and that defects in generating low affinity RFs might be an important trigger for the development of autoimmune diseases. The insulin data provided herein indicates that high affinity and low affinity IgM antibodies with opposite effects on their cognate antigen can develop against practically every autoantigen. Without being bound to theory, it can be assumed that the borderline between physiologic and pathologic autoimmunity is strongly marked by the affinity to the autoantigen rather than solely by tolerance mechanisms. Defects in establishing these equilibria may most likely lead to the development of autoimmune responses.

The inventors found that patients affected by autoimmune diseases have somewhat higher levels of total serum IgM and IgG antibodies as compared to healthy donors. However, the amount of RF-IgM detected in MS patients is significantly lower than the amount observed in healthy individuals. Thus, while RA patients are characterized by elevated amounts of high affinity protective RFs resulting in escalation of IgG function including autoreactive antibodies, MS patients most likely lack low affinity destructive RFs. The absence of low affinity RFs results in altered hemostasis of IgG antibodies leading to the accumulation and intensification of IgG function including autoreactive specificities.

Accordingly, the invention is at least in part based on the finding that the IgM antibody of the invention can be used to restore a high/low affinity RF ratio to prevent and/or treat an autoimmune disease.

In certain embodiments, the invention relates to the antibody for use of the invention, wherein the autoimmune disease or disorder is at least one selected from the group consisting of: systemic lupus erythematosus, rheumatoid arthritis and multiple sclerosis.

In certain embodiments, the invention relates to the antibody for use of the invention, wherein the autoimmune disease or disorder is rheumatoid arthritis.

RFs found in RA significantly differ from RFs found in healthy individuals as the latter are polyreactive and show little signs of affinity maturation. Conversely, RFs expressed by RA patients are highly somatically mutated, monospecific and possess high affinity for IgG.

In certain embodiments, the invention relates to a method for diagnosing an autoimmune disease or disorder, the method comprising the steps of: a) determining a high affinity rheumatoid factor portion and a low affinity rheumatoid factor portion based on the affinity of the rheumatoid factors to IgG antibodies in a sample, preferably an ex vivo sample, of a subject; and b) diagnosing the subject with an autoimmune disorder based on the high affinity rheumatoid factor portion and the low affinity rheumatoid factor portion determined in a) and/or a ratio thereof.

Based on the limited mutation rate and the reduced affinity, we propose that natural autoantibodies are primary IgM antibodies that are secreted in the course of early B cell activation before affinity maturation. In fact, earlier studies have shown that despite a similar use of V light and V heavy genes, RFs in healthy individuals show significantly reduced mutation patterns in their CDRs as compared with RFs of RA patients. Thus, the low affinity RFs found in healthy population are most likely the result of regulated selection mechanisms that limit affinity maturation in healthy individuals thereby preventing low affinity RF autoantibodies from becoming pathogenic.

In certain embodiments, the invention relates to the IgM antibody of the invention or the IgM antibody use of the invention, wherein the IgM antibody comprises: a variable heavy (VH) chain comprising CDR1 sequence as encoded by SEQ ID NO: 5, CDR2 sequence as encoded by SEQ ID NO: 6 and CDR3 sequence as encoded by SEQ ID NO: 7 and a variable light (VL) chain comprising CDR1 sequence as encoded by SEQ ID NO: 2, CDR2 sequence as encoded by GATGCATCC and CDR3 sequence as encoded by SEQ ID NO: 3.

In certain embodiments, the invention relates to the IgM antibody of claim 15 or the IgM antibody use of the invention, wherein the IgM antibody comprises: a variable heavy (VH) chain sequence comprising the amino acid sequence encoded by the sequence as defined by SEQ ID NO: 4 or by a sequence having at least 90% sequence identity to SEQ ID NO: 4, preferably at least 95% sequence identity to SEQ ID NO: 4; and a variable light (VL) chain sequence comprising the amino acid sequence encoded by the sequence as defined by SEQ ID NO: 1 or by a sequence having at least 90% sequence identity to SEQ ID NO: 1, preferably at least 95% sequence identity to SEQ ID NO: 1.

In certain embodiments, the invention relates to a host cell comprising a polynucleotide having 1) a) a sequence as defined by SEQ ID NO: 4 or a sequence having at least 90% sequence identity to SEQ ID NO: 4, preferably at least 95% sequence identity to SEQ ID NO: 4; and/or b) a sequence as defined by SEQ ID NO: 1 or a sequence having at least 90% sequence identity to SEQ ID NO: 1, preferably at least 95% sequence identity to SEQ ID NO: 1; and 2.) wherein the polynucleotide further encodes an IgM constant region and/or wherein the host cell comprises a further polynucleotide encoding an IgM constant region.

In certain embodiments, the invention relates to a method for producing an IgM antibody, the method comprising the steps of: a) culturing the host cell according to the invention; and b) isolating an IgM antibody.

“a,” “an,” and “the” are used herein to refer to one or to more than one (i.e., to at least one, or to one or more) of the grammatical object of the article.

“or” should be understood to mean either one, both, or any combination thereof of the alternatives.

“and/or” should be understood to mean either one, or both of the alternatives.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

The terms “include” and “comprise” are used synonymously. Unless the context requires otherwise, the term “comprise” or “include”, and variations such as “comprises/includes” and “comprising/including” etc., are to be understood in a non-exhaustive sense, i.e. they imply the inclusion but not exclusion of an element, integer, step or group thereof etc. By “consisting of” is meant including, and limited to, whatever follows the phrase “consisting of.”

“preferably” means one option out of a series of options not excluding other options. “e.g.” means one example without restriction to the mentioned example.

The terms “about” or “approximately”, as used herein, refer to “within 20%”, more preferably “within 10%”, and even more preferably “within 5%”, of a given value or range.

Reference throughout this specification to “one embodiment”, “an embodiment”, “a particular embodiment”, “a related embodiment”, “a certain embodiment”, “an additional embodiment”, “some embodiments”, “a specific embodiment” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The general methods and techniques described herein may be performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990).

While embodiments of the invention are illustrated and described in detail in the figures and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below.

### BRIEF DESCRIPTION OF FIGURES

FIG. 1: Recombinant low affinity anti-insulin IgM destructs insulin in vivo

A) Schematic representation of the recombinant in-house purified anti-insulin IGHV highlighting the two mutations in the CDR2 which were reverted to the germline version of the IGHV3-74*01 allele. bright gray: α-insulin IgM^{high} (WT-IGHV); medium gray: α-insulin IgM^{low} (gl-IGHV). B) Coomassie-stained SDS-PAGE showing purified α-insulin IgM^{high} and α-insulin IgM^{low} under reducing conditions (with β-mercaptoethanol). The image is representative of three independent experiments. C) Insulin-binding affinity of α-insulin IgM^{high} and α-insulin IgM^{low} measured by bio-layer interferometry. K_{D} (dissociation constant) was calculated by the software. The experiment shown is representative of 3 independent experiments. D) Blood glucose concentrations of WT mice intravenously (i.v.) injected with 100 μg α-insulin IgM^{high} (n=4) or α-insulin IgM^{low} (n=4) measured at indicated time points. Mean±SD, statistical significance was calculated using two-way ANOVA with Tukey's multiple comparison test. *p<0,05

FIG. 2: High affinity RF enhances the effect of autoreactive IgG

A) Blood glucose concentrations of WT mice intravenously injected with 100 μg anti-insulin IgG alone (n=4) or in combination with 20 μg RF concentrate from Rheumatoid Arthritis patients (RF^{high}, n=4) or monoclonal IgM control (mIgM, n=4) measured at indicated time points. Mean±SD, statistical significance was calculated using two-way ANOVA with Tukey's multiple comparison test. **p<0,01 B) Scheme depicting the procedure for isolation of total IgM from healthy donors (HD) sera. C) Coomassie-stained SDS-PAGE showing total IgM isolation from n=2 healthy donors (IgM^{HD}) under reducing conditions (with β-mercaptoethanol). The image is representative of three independent experiments. D) IgG-binding affinity of IgM isolated from healthy donors, RF^{high} and mIgM measured by bio-layer interferometry. K_{D} (dissociation constant) was calculated by the software. The experiment shown is representative of 3 independent experiments. E) Hep-2 slides showing anti-nuclear structure-reactive IgM (ANA) for total IgM isolation (IgM^{HD,}), RF^{high} and monoclonal IgM control. Scale bar 65 μm. Green fluorescence indicates IgM binding to Hep-2 cells. Images are representative of three independent experiments. F) Blood glucose concentrations of WT mice intravenously (i.v.) injected with 100 μg anti-insulin IgG combination with 20 μg total IgM purified from healthy donors (n=4) or with monoclonal IgM control (n=4) measured at indicated time points. Mean±SD, statistical significance was calculated using two-way ANOVA with Sidak's multiple comparison test. *p<0,01

FIG. 3: Recombinant low-affinity RF is polyreactive and binds DNA

A) Schematic representation of the immunoglobulin heavy and light variable genes (IGHV and IGLV, respectively) of the recombinant purified low-affinity RF as compared to the closest germline respective alleles. Mutations are highlighted bold.

IGHM: immunoglobulin heavy constant mu; IGVK: immunoglobulin variable kappa

B) Coomassie-stained SDS-PAGE showing recombinant monoclonal (in-house purified) low affinity RF (RF^{low}), commercial RF from Rheumatoid Arthritis patients (RF^{high}) and monoclonal control IgM (mIgM) under reducing conditions (with β-mercaptoethanol). The image is representative of three independent experiments. C) IgG-binding affinity of RF^{low} (purple line), RF^{high} (green line) and monoclonal IgM (blue line) measured by bio-layer interferometry. K_{D} (dissociation constant) was calculated by the software. The experiment shown is representative of 3 independent experiments. D) Anti-IgG IgM concentrations detected in purified RF^{low} (n=3), RF^{high} (n=3) and mIgM control (n=3) measured by ELISA (coating: human IgG) Mean±SD, statistical significance was calculated using ordinary one-way ANOVA with Tukey's multiple comparisons test. **p<0,01 E) Anti-dsDNA-IgM concentrations of RF^{low} (n=3), RF^{high} (n=3) and IgM control (n=3) measured by ELISA (coating: calf-thymus dsDNA). Mean±SD. Results are representative of three independent measurements. F) Hep-2 slides showing anti-nuclear structure-reactive IgM (ANA). Scale bar 65 μm. Green fluorescence indicates IgM binding to Hep-2 cells. Images are representative of three independent experiments. G) Schematic summary of the characteristics of RF^{high} and RF^{low}.

FIG. 4: RF^{low} controls IgG in vivo function by enhanced degradation

A Blood glucose concentrations of WT mice intravenously (i.v.) injected with 100 μg anti-insulin IgG alone (n=4) or in combination with 20 μg RF^{low} (n=4) or mIgM control (n=4) measured at indicated time points. Mean±SD, statistical significance was calculated using two-way ANOVA with Tukey's multiple comparison test. **p<0,01

B Serum human IgG concentrations of WT mice at day 0 and day 1 after a single i.v. injection of 20 μg of α-CD20 human IgG (Rituximab) alone (n=4) or in combination with RF^{high} (n=4) or mIgM control (n=4) as measured by ELISA. Mean±SD, statistical significance was calculated using two-way ANOVA with Tukey's multiple comparison test. ****p<0,0001

C Serum human IgG concentrations of WT mice at day 0 and day 1 after a single i.v. injection of 20 μg α-CD20 human IgG (Rituximab) in combination with RF^{high} (n=4), with RF^{low} (n=4) or IgM ctrl (n=5) as measured by ELISA. Mean±SD, statistical significance was calculated using two-way ANOVA with Tukey's multiple comparison test. *p<0,05; ****p<0,0001

FIG. 5: RF^{low} prevails over RF^{high}

A Blood glucose concentrations of WT mice intravenously (i.v.) injected with 100 μg anti-insulin IgG in combination with 20 μg RF^{high} (n=7), 20 μg RF^{low} (n=6), 20 μg RF^{low} ⁺20 μg RF^{high} (n=7) or IgM control (n=5) measured at indicated time points. Mean±SD, statistical significance was calculated using two-way ANOVA with Tukey's multiple comparison test. *p<0,05; **p<0,01; ***p<0,001; ****p<0,0001

FIG. 6: Deregulated ratios of high affinity and low affinity RFs in autoimmune diseases

A Total IgM amount detected in serum from young (n=20,) and aged (n=17) healthy donors (HD), Rheumatoid Arthritis (RA) patients (n=15) and Multiple Sclerosis (MS) patients (n=28, red bar) measured by ELISA. Bars depict mean±SD, individual values are represented by single dots. Statistical significance was calculated using Kruskal Wallis test. *p<0,05; **p<0,01

Mean values of IgM (μg/ml) as follows: Young HD 1517,55; Aged HD 1258,02; MS patients 2143,72; RA patients 2361,29.

B Total IgG amount detected in serum from young (n=20) and aged (n=17) healthy donors (HD), Rheumatoid Arthritis (RA) patients (n=15) and Multiple Sclerosis (MS) patients (n=28) measured by ELISA. Bars depict mean±SD, individual values are represented by single dots. Statistical significance was calculated using Kruskal Wallis test. **p<0,01

Mean values of IgG (μg/ml) as follows: Young HD 7733,22; Aged HD 6856,48; MS patients 10419,28; RA patients 10345,23.

C Total RF-IgM detected in serum from young (n=20) and aged (n=17) healthy donors (HD), Rheumatoid Arthritis (RA) patients (n=15) and Multiple Sclerosis (MS) patients (n=28) measured by ELISA (coating: human IgG). Bars depict mean±SD, individual values are represented by single dots. Values from RA patients plotted separately for simplified visualization. Statistical significance was calculated using Kruskal Wallis test. *p<0,05; **p<0,01; ****p<0,0001

Mean values of RF-IgM (AU) as follows: Young HD 4,71; Aged HD 2,31; MS patients 1,72; RA patients 737,58.

FIG. 7:

A Anti-Insulin-IgM concentrations detected in recombinant in-house purified anti-Insulin IgM^{high} (WT, n=3) and anti-Insulin IgM^{low} (gl, n=3) as measured by ELISA (coating: human Insulin). Mean±SD depicted. Data are representative of three independent measurements.

FIG. 8:

A Kinetic plot showing mean±SD of blood glucose levels after injection of 100 μg anti-Insulin IgG (black line, n=5) or IgG isotype control (n=5). Statistical significance was calculated using two-way ANOVA with Sidak's multiple comparison test. **p<0,01

B IgG-binding IgM concentrations in RF-IgM elution from healthy donors (RF-IgM^{HD}, n=3) and from RA patients (RF-IgM^{RA}, n=3) as measured by ELISA (coating: human IgG). Mean±SD, statistical significance was calculated using unpaired t test. *p<0,05.

C IgG-binding affinity of RF-IgM isolated from healthy donors and from RA patients measured by bio-layer interferometry. K_{D} (dissociation constant) was calculated by the software. The experiment shown is representative of 3 independent experiments.

D IgG-binding IgM concentrations in total IgM isolated from healthy donors (n=3) compared with IgG-binding IgM amount detected in RF^{high} (n=3) and in monoclonal IgM (n=3) as measured by ELISA (coating: human IgG). Mean±SD, statistical significance was calculated using ordinary one-way ANOVA with Tukey's multiple comparisons test. ***p<0,001

### EXAMPLES

### Example 1: Recombinant Low Affinity Anti-Insulin IgM Destructs Insulin In Vivo

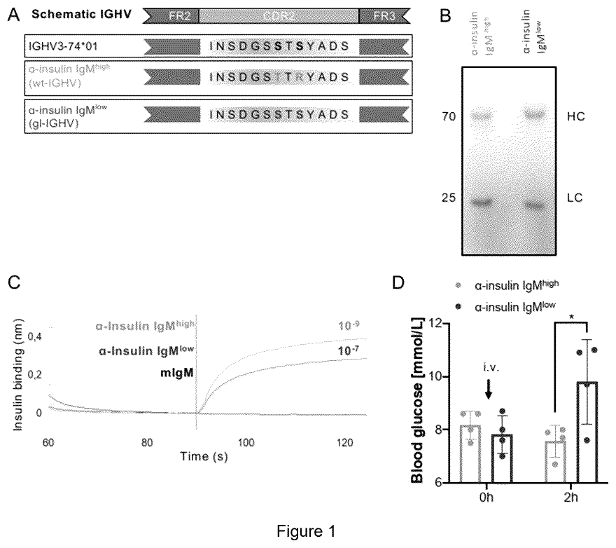

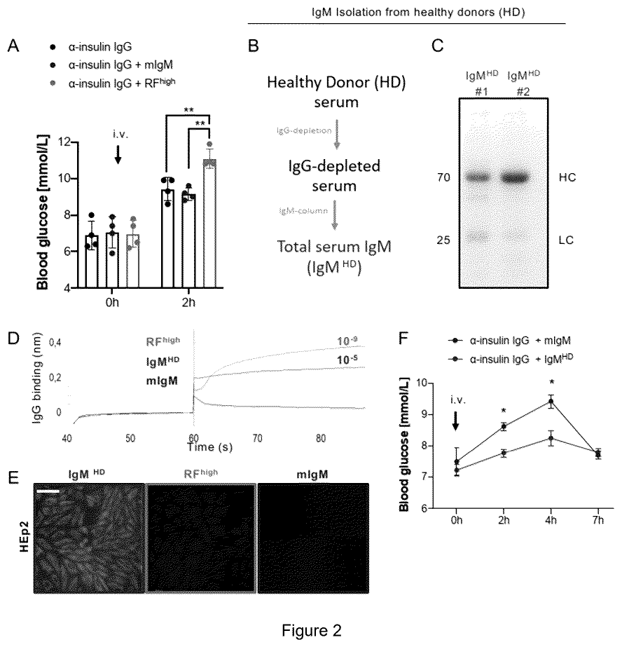

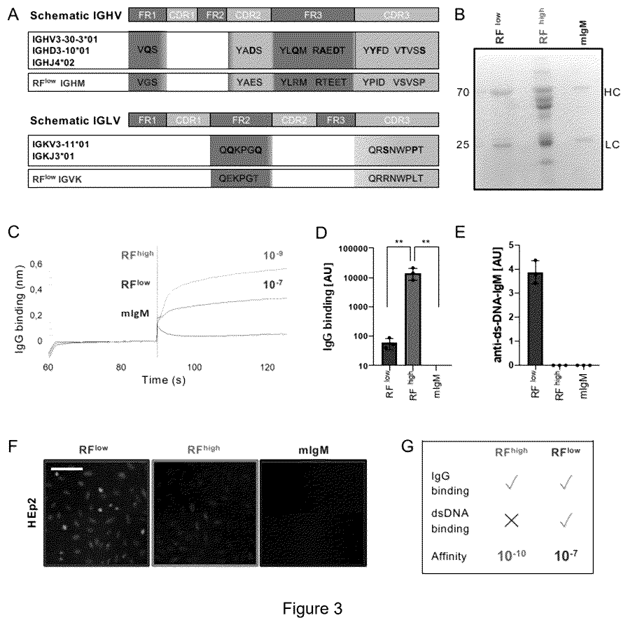

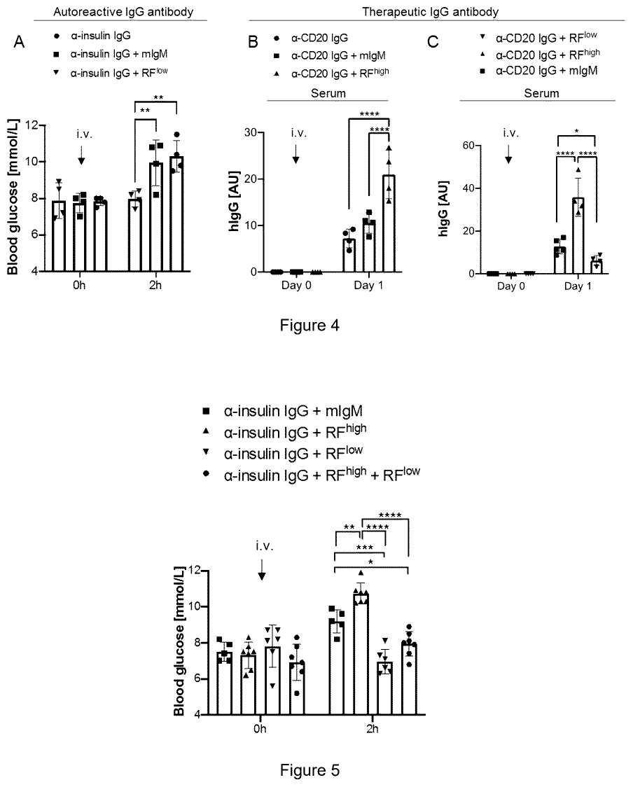

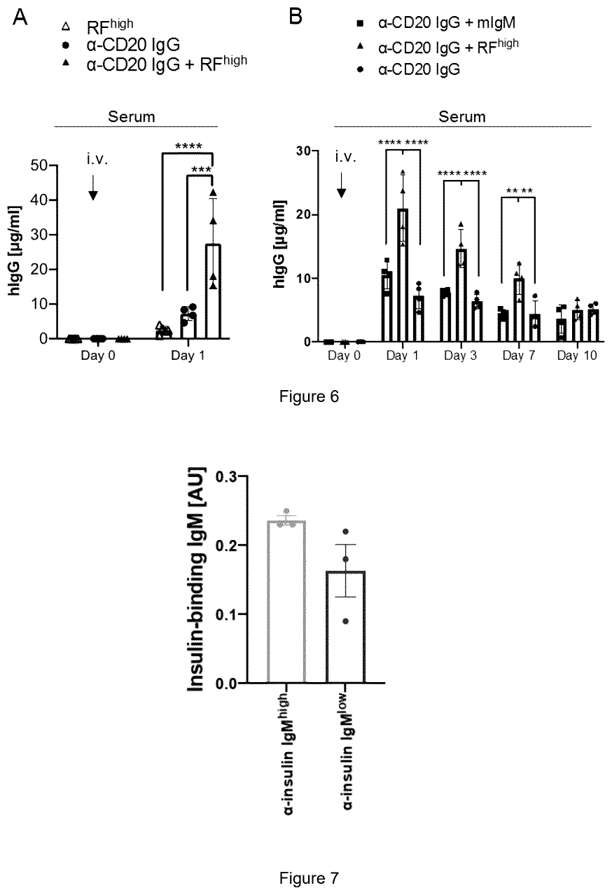

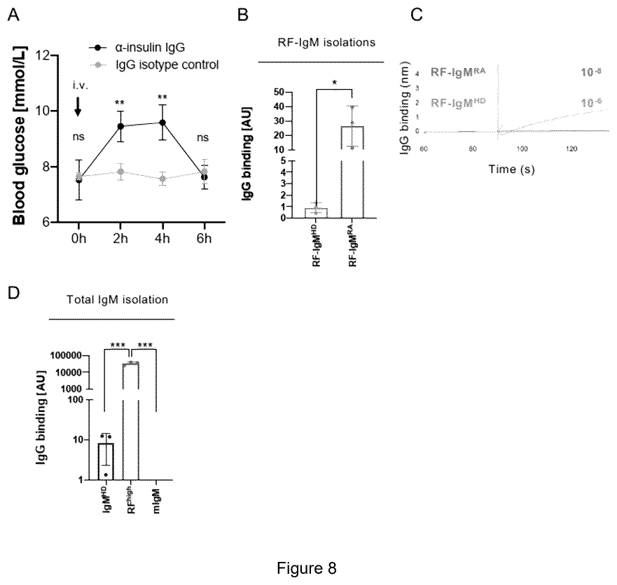

To confirm our hypothesis that IgM affinity and specificity determines the outcome of the interaction with the recognized cognate antigen, we used recombinant anti-insulin antibodies as model. Since we proposed that affinity to the target and mono-specificity are the main requirements for determining the effector function of autoreactive antibodies, we expect that reversion of the variable region of the anti-insulin IgM into its respective germline (gl) version would result in reduced affinity to its target. To this end, we reverted the heavy chain (HL) and the light chain (LC) sequences to germline and tested combinations of the reverted HC/LC for their insulin binding affinity. While most combinations lost insulin binding, the recombinant insulin-specific antibody (anti-insulin IgM^{low}) consisting of the original LC and the germline-reverted HC version of the anti-insulin antibody showed reduced affinity to insulin as compared with the original antibody (FIG. 1A). In fact, the K_{D} of the germline-reverted anti-insulin IgM^{low} was in the range of 10⁻⁷ (FIG. 1C) and thus, considerably lower than the affinity of the original anti-insulin IgM^{high}. In addition, decreased insulin binding was observed for anti-insulin IgM^{low} by ELISA (FIG. 7). In order to test whether the two antibodies, namely anti-insulin IgM^{low} and its high affinity counterpart IgM^{high}, had different effects on glucose metabolism, we injected identical molar amounts of anti-insulin IgM^{high} and anti-insulin IgM^{low} into WT mice. Within two hours after injections, higher blood glucose levels (hyperglycemia) were observed in mice that received anti-insulin IgM^{low}, while anti-insulin IgM^{high} did not alter blood glucose and was able to protect insulin from IgG-dependent degradation (FIG. 1D).

Interestingly, the reverted version of the anti-insulin IgM differs in only two point mutations in the complementarity-determining region 2 (CDR2) that seem to be responsible for the affinity maturation (FIG. 1A). Importantly, the quality of the in vitro produced antibodies was assessed and revealed no structural difference between the purified IgM^{high} and the IgM^{low} antibodies (FIG. 1B).

These data suggest that a high affinity autoantibody with a protective role can be turned into an autoantibody with a destructive role by reverting the immunoglobulin heavy chain variable region (IGHV) into its germline version (low affinity). This confirms our hypothesis of the regulatory role of IgM antibodies and suggests that mutations acquired during the affinity maturation process can turn destructive IgM antibodies into protective ones.

### Example 2: Recombinant Low-Affinity RF is Polyreactive and Binds DNA

In order to confirm our findings regarding the role of low affinity RF in the interaction with the target antigen, we reviewed available reports describing the extent of somatic mutations of RFs in rheumatoid arthritis (RA) patients (Randen et al. 1992; Youngblood et al. 1994). Albeit the majority of rheumatoid factors (RFs) isolated from the synovia of RA patients are highly affine for the Fc portion of IgG and not reactive to other tested antigens, we identified one RF (RF-IgM) isolated from a RA patient that seemed to be polyreactive and binds to other antigens, such as tetanus toxoid, DNA and bovine serum albumin (BSA) (Youngblood et al. 1994). Interestingly, in-depth analysis of the IGHV and IGLV sequences of the selected RF revealed high degree of homology to the germline gene counterparts. In fact, the selected antibody variable heavy chain shared 96,9% identical residues with the IGHV3-30-3*01 (allele 1) and the light chain had 99,3% identity with the IGKV3-11*01 (FIG. 3A). Due to the high degree of identity to germline genes and to the previously published data showing the polyreactivity of this RF, we expected this antibody to be low affinity RF (RF^{low}). Therefore, we cloned and expressed RF^{low} as recombinant IgM (FIG. 3B). Bio-layer interferometry assay revealed that the IgG-binding affinity of RF^{low} was in the range of 10⁻⁷, while the K_{D} of RF^{high} was 10⁻⁹ (FIG. 3C).

The ability of RF^{low} to bind IgG was also tested by ELISA revealing that the recombinant RF^{low} binds IgG although to a lesser extent than RF^{high} which is most likely a result of the reduced IgG affinity of RF_{low} (FIG. 3D). Additionally, we confirmed the previously published data showing that, in contrast to RF^{high}, recombinant RF^{low} binds double-stranded DNA (FIG. 3E) and is reactive in HEp2 slides (FIG. 3F).

These data confirm available data suggesting that, in contrast to typical high affinity RFs from RA patients, low affinity RFs are multi-specific/poly-reactive as they bind DNA in addition to IgG (FIG. 3G).

### Example 3: RF^{low} Controls IgG in Vivo Function by Enhanced Degradation

Using the above described monoclonal low affinity RF (RF^{low}), we tested whether a low affinity RF would neutralize its target in vivo. To this end, we injected WT mice with anti-insulin IgG together with equal molar amounts of RF^{low} or with a non-specific monoclonal IgM as control (mIgM). As expected, mice injected with anti-insulin IgG only or with anti-insulin IgG together with control mIgM showed comparable increase in blood glucose levels. In contrast, mice which received RF^{low} along with the anti-insulin IgG showed constant blood glucose level suggesting that RF^{low} controls the function of autoreactive IgG (FIG. 4A).

Next, we investigated whether the protective or destructive effect of RF could be observed with other IgGs such as therapeutic antibodies. To this end, we used Rituximab as a well-known therapeutic IgG antibody targeting CD20. This monoclonal anti-CD20 antibody, which consists of human constant regions and murine variable domains (Pierpont, Limper, and Richards 2018; Tobinai 2001), is approved for treatment of B cell malignancies as well as autoimmune diseases such as RA and Systemic Lupus erythematosus (SLE) (Aletaha and Smolen 2018; Malmström et al. 2017; Taylor and Lindorfer 2007). We intravenously injected into WT mice equal molar amounts of anti-CD20 IgG either alone or combined with RF^{high} or with mIgM and we monitored human IgG (hIgG) concentrations over time. Our data showed that mice injected with Rituximab together with RF^{high} exhibited significantly higher levels of hIgG as compared to mice that received anti-CD20 IgG alone or in combination with mIgM (FIG. 4B). Together with the data above, these results led us to the hypothesis that if the higher hIgG titer was due to the presence of RF^{high}, the co-injection of RF^{low} with anti-CD20 IgG should show opposite effect, i.e. hIgG level reduction over time. Therefore, we injected anti-CD20 IgG combined with equal molar amounts of the recombinant RF^{low} or of the control monoclonal mIgM. We observed a significant difference in the hIgG level between the two groups already one day after injection. In fact, the animals injected with RF^{low} along with anti-CD20 IgG showed significantly lower concentration of hIgG in respect to the mice which received RF^{high} (FIG. 4C).

Our results show that a high affinity RF is capable of stabilizing IgG in vivo thereby impressively extending its half-life, while a low affinity RF exhibit the opposite destructive effect in vivo. Together, these data indicate that RFs have different impact on the half-life of IgG depending on their affinity to their target. Interestingly, this is not only valid for autoreactive antibodies but also for therapeutic antibodies.

### Example 4: RF^{low} Prevails Over RF^{high}

In order to better understand the dynamic of RFs interaction with IgG in vivo, we investigated the effects of the combined presence of low and high affinity RFs on IgG function. To this end, we injected into WT mice anti-insulin IgG with equal molar amounts of RF^{high} and RF^{low} and subsequently, we monitored blood glucose levels. As expected, the blood glucose levels of the mice injected with anti-insulin IgG combined with mIgM were increased within two hours after injection. Intriguingly, the blood glucose levels of the mice injected with the insulin-specific IgG and the combination of RF^{high} and RF^{low} (anti-insulin IgG+RF^{high}+RF^{low}) did not differ from the levels of mice injected with anti-insulin IgG with RF^{low} only, suggesting that RF^{high} cannot exert its protective role in the presence of RF^{low}. In fact, blood glucose concentrations in these mice, which received anti-insulin IgG+RF^{high}+RF^{low}, were significantly lower than the concentrations in mice which received anti-insulin IgG with RF^{high} alone (FIG. 5).

In summary, our data suggest that the presence of RF^{low} counteracts the stabilizing activity of RF^{high} resulting in target destruction which is comparable to the effect observed with RF^{low} only.

### Example 5: Deregulated Ratios of High Affinity and Low Affinity RFs in Autoimmune Diseases

The above results suggesting that the effects observed in the presence of a low affinity RF prevails over the effects of a high affinity RF lead us to the hypothesis that a failure in maintaining the balance between the two classes of RFs might contribute to the development of autoimmune diseases. To gain a deeper understanding, we collected sera from young and aged healthy donor and from patients suffering from two well-known autoimmune diseases, namely RA and multiple sclerosis (MS). We characterized these samples for total serum levels of IgM and IgG. Interestingly, total serum IgM levels of MS and RA patients seem to be increased as compared with healthy individuals (FIG. 6A). Furthermore, while total serum IgG concentration of young and aged healthy individuals were in similar range, the IgG levels of MS patients were significantly increased as compared to aged healthy individuals and a similar, although not significant, tendency was shown by total IgG levels of RA patients (FIG. 6B).

Next, the inventors assessed whether the higher circulating levels of IgG in MS correlate with altered amounts of circulating RF-IgM. Interestingly, MS patients show significantly lower amounts of RF-IgM than the young and aged healthy individuals (FIG. 6C). These data suggest that low affinity RF is reduced in MS patients as compared with healthy individuals and, therefore, it is conceivable that the regulation of IgG homeostasis including autoreactive antibodies is altered.

Altogether, these findings suggest that an increase in IgG-protective RF^{high} in RA patients or a decrease in IgG-destructive RF^{low} in MS patients might be important pathogenic mechanisms associated with the development of autoimmune diseases.

### Mice

8- to 15-week-old female C57BL/6 mice were used in all experiments reported in this study. For antibody stability experiment, 20-50 μg antibodies (as indicated in details in figure legend for each experiment) were injected intravenously (i.v.) into the lateral tail vein and blood was collected at indicated time points to obtain serum.

For blood glucose monitoring experiments 100 μg anti-insulin IgG or anti-insulin IgM were injected i.v. into the lateral tail vein and blood was collected at indicated time points to obtain serum.

Animal experiments were performed in compliance with the guidelines of German law and approved by the responsible regional board Tübingen, Germany under the license 1484. All mice used in this study were either bred and housed within the animal facility of Ulm University under specific-pathogen-free conditions or obtained from Charles River at the age of 6 weeks.

Antibody specificity, host/isotype, conjugate clone, class, supplier catalog number:

Anti-human CD20 (Rituximab, human IgG1, SelleckChem); Rheumatoid Factor Concentrate (Lee Biosolutions), Human IgM (unlabeled, SouthernBiotech, #0158L-01), RF^{low} (human IgM, homemade with a IgM constant region, sequence of heavy chain and light chain from Youngblood, Kathy, Lori Fruchter, Guifeng Ding, Javier Lopez, Vincent Bonagura, and Anne Davidson. 1994. Journal of Clinical Investigation 93(2):852-61. —RC1 having a VH sequence as encoded by the sequence defined by SEQ ID NO: 4 (HDCR1 encoded by the sequence defined by SEQ ID NO: 5, HCDR2 encoded by the sequence defined by SEQ ID NO: 6, HCDR2 encoded by the sequence defined by SEQ ID NO: 7) and a VL sequence as encoded by the sequence defined by SEQ ID NO: 1 (LDCR1 encoded by the sequence defined by SEQ ID NO: 2, LCDR2 encoded by GATGCATCC, LCDR2 encoded by the sequence defined by SEQ ID NO: 3); RF^{high} (human IgM, homemade with a IgM constant region, sequence of heavy chain and light chain from Youngblood, Kathy, Lori Fruchter, Guifeng Ding, Javier Lopez, Vincent Bonagura, and Anne Davidson. 1994. Journal of Clinical Investigation 93(2):852-61. —RO7 having a VH sequence as encoded by the sequence defined by SEQ ID NO: 11 (HDCR1 encoded by the sequence defined by SEQ ID NO: 12, HCDR2 encoded by the sequence defined by SEQ ID NO: 13, HCDR2 encoded by the sequence defined by SEQ ID NO: 14) and a VL sequence as encoded by the sequence defined by SEQ ID NO: 8 (LDCR1 encoded by the sequence defined by SEQ ID NO: 9, LCDR2 encoded by GGTGCATCC, LCDR2 encoded by the sequence defined by SEQ ID NO: 10).

anti-Insulin IgG (purified from IVIg, see below); total serum IgM (isolated from healthy donor serum, see below); anti-insulin IgM^{high} and anti-insulin IgM^{low} (human IgM, homemade, sequence from Ikematsu, H., Y. Ichiyoshi, E. W. Schettino, M. Nakamura, and P. Casali. 1994. Journal of Immunology 152(3):1430-41., germline reversion was achieved using the online available tool IMGT® V-Quest).

### HEK293-6E Cell Culturing and Antibody Production

HEK293-6E cells were cultured in FreeStyle F17 expression media (Invitrogen) supplemented with 0,1% Kolliphor® P188 (Sigma-Aldrich) and 4 mM L-Glutamine (Gibco Life Technologies). Transfection was performed according to the manufacturer's instructions. Briefly, cells were transfected using Polyethylenimine (Polysciences) with two pTT5 plasmids encoding heavy and light chain of the antibody of interest (total 1 μg DNA/ml of culture). 24-48 hours post transfection cells were fed with Tryptone N1 (TekniScience Inc #19553) to a final concentration of 0,5%.

Harvesting was performed 120 hours post transfection. Antibodies were purified using HiTrap® IgM columns (GE Healthcare, Sigma-Aldrich) as described below.

### Antibody Purification and Pulldown of Total Serum IgM

For IgM purification from human serum, IgG depletion was performed by incubating the samples with Protein G Sepharose beads (GE Healthcare, Sigma-Aldrich) according to manufacturer's instructions.

For IgM purification from IgG-depleted human serum and from HEK293-6E cell supernatant, HiTrap® IgM columns (GE Healthcare, Sigma-Aldrich) were used according to the manufacturer's protocol and eluates were dialyzed overnight in 300-fold sample volume 1x PBS. Quality control of the isolated immunoglobulins was addressed via SDS-PAGE stained with Coomassie-brilliant blue R-250 (BIO-RAD) and the quantification of eluted proteins was assessed via ELISA.

### Isolation of Antigen-Specific Immunoglobulins From IVIg

Streptavidin bead columns (Thermo Scientific, #21115) were loaded with 20 μg biotin-insulin (ibt biosystem). IVIg preparation was incubated for 90 min at room temperature to ensure binding of antigen-specific antibodies to the beads. Isolation of the antibodies was performed by acidic pH-shift using the manufacturer's elution and neutralization solutions. Quality of the isolated immunoglobulins was examined via SDS-PAGE stained with Coomassie-brilliant blue R-250 (BIO-RAD) and ELISA. For further in vivo experiments, the isolated antibodies were dialyzed overnight in 300-fold sample volume 1x PBS.

### Enzyme-Linked Immunosorbent Assay (ELISA)

96-Well plates (Nunc, ThermoScientific) were coated either with 10 μg/ml anti-human IgM or anti-human IgG-antibodies (SouthernBiotech) or with 10 μg/ml human IgG (SouthernBiotech) or with 2,5 μg/ml calf thymus dsDNA (Rockland) or with 2,5 μg/ml native insulin (Sigma-Aldrich). Blocking was done in 1% BSA blocking buffer (SERVA). Serial dilutions of 1:3 IgM or IgG antibodies (SouthernBiotech) were used as standard. The relative concentrations stated as arbitrary unit (AU), were determined via detection by Alkaline Phosphatase (AP)-labeled anti-IgM/anti-IgG (SouthernBiotech). The p-nitrophenylphosphate (pNPP; Genaxxon) in diethanolamine buffer was added and data were acquired at 405 nm using a Multiskan FC ELISA plate reader (Thermo Scientific). All samples were measured in duplicates.

Antibody specificity, host/isotype, conjugate clone, class, supplier catalog number: anti-human IgM (goat, IgG, unlabeled, polyclonal, SouthernBiotech, #2020-01); anti-human IgG (goat, IgG, unlabeled, polyclonal, SouthernBiotech, #2040-01); human IgM (unlabeled, SouthernBiotech, #0158L-01); human IgG (unlabeled, SouthernBiotech, #0150-01); anti-human IgM (mouse, AP, monoclonal, SouthernBiotech, #9020-04); anti-human IgG (Goat, AP, polyclonal, SouthernBiotech, #2040-04).

### HEp-2 Slides and Fluorescence Microscopy

Kallestad HEp-2 slides (BIO-RAD, #26101) were used to assess reactivity of purified homemade IgM or pulldown serum IgM to nuclear antigens (ANA). Approximately 10 μg per sample were applied onto the HEp-2 slides. Anti-IgM-FITC (Biolegend, #314506) was used for detection of ANA-IgM. Stained HEp-2 slides were analyzed using fluorescence microscope DMi8 (Leica) and Leica Application Suite X (LAS X) software (Leica).

### Monitoring of Blood and Urine Glucose Levels

AccuChek (Roche Diagnostics, Mannheim) blood glucose monitor was used to measure blood glucose levels of mice. Blood was taken from the lateral tail vein from ad libitum fed mice and transferred onto sterile test stripes. Glucose levels were measured in mmol/l at hours stated in the figures for each mouse per group.

### SDS-PAGE, Coomassie

Samples were separated on 10-12% SDS-polyacrylamide gels incubated with Coomassie-brilliant blue R-250 (BIO-RAD) for 45 min and subsequently de-stained.

### Healthy Donors and Patients Samples

Healthy donor blood samples were obtained via the Deutsch Rotes Kreuz Ulm (DRK). Samples were divided into young (18-35 years) and old (above 55 years old) according to their age. Sera was obtained by Pancoll gradient centrifugation.

Sera from Multiple Sclerosis patients were provided by the Biobank of the Rehabilitationskrankenhaus of the University Hospital Ulm (RKU).

Sera from Rheumatoid Arthritis (RA) patients were provided by the Clinic of Rheumatology and clinical Immunology of the University Clinic of Freiburg. RA patients were categorized according to symptoms and positivity for RF.

### Bio-Layer Interferometry (BLI)

Bio-Layer Interferometric assays (BLItz device, FortéBio) were used to determine the affinity of antigen-antibody interactions (Kumaraswamy, Sriram, and Renee Tobias. 2015. “Label-Free Kinetic Analysis of an Antibody-Antigen Interaction Using Biolayer Interferometry.” Pp. 165-82 in). Here, we used insulin-specific IgM or RF-IgM and insulin-bio (ibt biosystem) or human IgG-bio (labeled using LYNX Rapid Biotin Antibody conjugation kit, BIORAD) as targets. Targets were loaded onto streptavidin biosensors (FortéBio). Binding affinities of IgM to insulin or IgG were acquired in relative wavelength shift in nm. Subsequently, the calculated affinity value (Kₐ) was used to determine the dissociation constant (K_{D}): K_{D}=1/Kₐ. The following protocol was used during the measurements: 30-s baseline, 30-s loading, 30-s baseline, 120-s association, 60-s dissociation. For buffering of samples, targets, and probes, the manufacturer's sample buffer (FortéBio) was used.

1. A glycosylated IgM antibody cross-specifically binding to an IgG antibody and to a complexing molecule, wherein the binding to the IgG antibody and the complexing molecule induces degradation of the IgG antibody.

2. The antibody according to claim 1, wherein the K_{d} for the binding affinity of the IgM antibody to the IgG antibody is in the range of 10⁻⁵ to 10⁻⁸, preferably 10⁻⁷.

3. The antibody according to claim 1 or 2, wherein at least one complementarity-determining region (CDR) of the IgM antibody binds to the IgG antibody.

4. The antibody according to the preceding claims, wherein glycosylated part of the IgM antibody binds to the complexing molecule.

5. The antibody according to any one of the preceding claims, wherein the IgG antibody is an autoreactive IgG antibody.

6. The antibody according to any one of the preceding claims, wherein the complexing molecule is a nucleic acid, preferably DNA.

7. The antibody according to any one of the preceding claims, wherein the autoreactive IgG antibody is an anti-citrullinated protein-IgG antibody.

8. The antibody according to any one of the preceding claims, wherein a first chain of the IgM antibody comprises CDRs specifically binding to IgG and a second chain of the IgM antibody comprises CDRs polyreactively binding to IgG.

9. The antibody according to any one of the preceding claims, for use in medicine.

10. The antibody according to any one of claims 1 to 8, for use in the treatment of a subject with increased IgM level, preferably a serum IgM level above 1500 hIgM μm/ml.

11. The antibody according to any one of claims 1 to 8, for use in the treatment of a subject with an increased high affinity rheumatoid factor: low affinity rheumatoid factor ratio.

12. The antibody according to any one of claims 1 to 8, for use in the treatment of an autoimmune disease or disorder, preferably a chronic autoimmune disease or disorder.

13. The antibody for use of claim 12, wherein the autoimmune disease or disorder is at least one selected from the group consisting of: Systemic lupus erythematosus, rheumatoid arthritis and multiple sclerosis.

14. The antibody for use of claim 13, wherein the autoimmune disease or disorder is rheumatoid arthritis.

15. A method for diagnosing an autoimmune disease or disorder, the method comprising the steps of:
a) determining a high affinity rheumatoid factor portion and a low affinity rheumatoid factor portion based on the affinity of the rheumatoid factors to IgG antibodies in a sample of a subject; and
b) diagnosing the subject with an autoimmune disorder based on the high affinity rheumatoid factor portion and the low affinity rheumatoid factor portion determined in a) and/or a ratio thereof.

16. The IgM antibody of any one of claims 1 to 8 or the IgM antibody for use of any of claims 9 to 14, wherein the antibody comprises:
a variable heavy (VH) chain comprising CDR1 sequence as encoded by SEQ ID NO: 5, CDR2 sequence as encoded by SEQ ID NO: 6, and CDR3 sequence as encoded by SEQ ID NO: 7, and
a variable light (VL) chain comprising CDR1 sequence as encoded by SEQ ID NO: 2, CDR2 sequence as encoded by GATGCATCC and CDR3 sequence as encoded by SEQ ID NO: 3.

17. The IgM antibody of claim 16 or the IgM antibody for use of claim 16, wherein the antibody comprises:
a variable heavy (VH) chain sequence comprising the amino acid sequence encoded by the sequence as defined by SEQ ID NO: 4 or by a sequence having at least 90% sequence identity to SEQ ID NO: 4, preferably at least 95% sequence identity to SEQ ID NO: 4; and
a variable light (VL) chain sequence comprising the amino acid sequence encoded by the sequence as defined by SEQ ID NO: 1 or by a sequence having at least 90% sequence identity to SEQ ID NO: 1, preferably at least 95% sequence identity to SEQ ID NO: 1.

18. A host cell comprising a polynucleotide having
a) a sequence as defined by SEQ ID NO: 4 or a sequence having at least 90% sequence identity to SEQ ID NO: 4, preferably at least 95% sequence identity to SEQ ID NO: 4; and/or
b) a sequence as defined by SEQ ID NO: 1 or a sequence having at least 90% sequence identity to SEQ ID NO: 1, preferably at least 95% sequence identity to SEQ ID NO: 1,
wherein preferably the polynucleotide further encodes an IgM constant region and/or wherein preferably the host cell comprises a further polynucleotide encoding an IgM constant region.

19. A method for producing an IgM antibody, the method comprising the steps of:
a) culturing the host cell according to claim 18, and
b) isolating an IgM antibody.


SEQUENCE LISTING (14 sequences)

SEQ ID NO: 1 - DNA, 381 bp, synthetic construct
  source 1..381: mol_type = other DNA
atggaagccccagctcagcttctcttcctcctgctactctggctcccagataccaccggagaaattgtgttgacacagtctccagccaccctgtctttgtctccaggggaaagagccaccctctcctgcagggccagtcagagtgttagcagctacttagcctggtaccaagagaaacctggaacggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtaggaactggcctctcactttcggccctgggaccaaagtggatatcaaa

SEQ ID NO: 2 - DNA, 18 bp, synthetic construct
  source 1..18: mol_type = other DNA
cagagtgttagcagctac

SEQ ID NO: 3 - DNA, 27 bp, synthetic construct
  source 1..27: mol_type = other DNA
cagcagcgtaggaactggcctctcact

SEQ ID NO: 4 - DNA, 435 bp, synthetic construct
  source 1..435: mol_type = other DNA
atggagtttgggctgagctgggttttcctcgttgctcttttaagaggtgtccagtgtcaggtgcagctggtgcagtcggggggaggcgtggtccagcctgggaggtccctgagactctcctgtgcagcctctggattcaccttcagtagctatgctatgcactgggtccgccaggctccaggcaaggggctggagtgggtggcagttatatcatatgatggaagcaataaatactacgcagagtccgtgaagggccgattcaccatctccagagacaattccaagaacacgctgtatctgcgaatgaacagcctgagaactgaggaaacggctgtgtactactgtgcgagagacggtgatgtgggttcctatgggccggggacctattatcctattgactactggggccagggaaccctggtctccgtctcccca

SEQ ID NO: 5 - DNA, 24 bp, synthetic construct
  source 1..24: mol_type = other DNA
ggattcaccttcagtagctatgct

SEQ ID NO: 6 - DNA, 24 bp, synthetic construct
  source 1..24: mol_type = other DNA
atatcatatgatggaagcaataaa

SEQ ID NO: 7 - DNA, 57 bp, synthetic construct
  source 1..57: mol_type = other DNA
gcgagagacggtgatgtgggttcctatgggccggggacctattatcctattgactac

SEQ ID NO: 8 - DNA, 384 bp, synthetic construct
  source 1..384: mol_type = other DNA
atggaaaccccagcgcagcttctcttcctcctgctactctggctcccagataccaccggagaaattgtgttgacgcagtctccaggcaccctgtctttgtctccaggggaaagagccaccctctcctgcagggccagtcagagtgtcagcagcacctactttgcctggtaccagcagaaacctggccaggctcccaggctcctcatctatggtgcatccaacagggccactggcatcccagacaagttcaatggcagtgggtctgggacagacttcactctcaccatcagtagactggagcctgaagattttgcagtgtattactgtcagcagtatggtagttcaccgctcactttcggcggagggaccaaggtggagatcaga

SEQ ID NO: 9 - DNA, 21 bp, synthetic construct
  source 1..21: mol_type = other DNA
cagagtgtcagcagcacctac

SEQ ID NO: 10 - DNA, 27 bp, synthetic construct
  source 1..27: mol_type = other DNA
cagcagtatggtagttcaccgctcact

SEQ ID NO: 11 - DNA, 426 bp, synthetic construct
  source 1..426: mol_type = other DNA
atggactggacctggaggttcctctttgtggtggcagcagctacaggtgtccagtcccaggtgcaactggtgcagtctggggctgaggtgaagaagcctgggtcctcggtgaaggtctcgtgcaaggcttctggaggcactttcgacagatatactataaactgggtgcgacattcccctggacaagggcttgagtggatgggagggatcatccctatgtttggcacaccgaactacgcagagaagtttcggggcagagtcgcgatttccgcggagaaatccacgaccacagcctacatggaaatgagcagcctaacatctgaggacacggccatatattactgtgcgcgagggtcgggggaacacacaaatatggtggtgccctttgactactggggccagggaaccctggtcaccgtctcctca

SEQ ID NO: 12 - DNA, 24 bp, synthetic construct
  source 1..24: mol_type = other DNA
ggaggcactttcgacagatatact

SEQ ID NO: 13 - DNA, 24 bp, synthetic construct
  source 1..24: mol_type = other DNA
atcatccctatgtttggcacaccg

SEQ ID NO: 14 - DNA, 48 bp, synthetic construct
  source 1..48: mol_type = other DNA
gcgcgagggtcgggggaacacacaaatatggtggtgccctttgactac